(19) United States
(12) Patent Application Publication
(10) Pub. No.: US 2008/0095818 A1
(43) Pub. Date: Apr. 24, 2008

(54) Tubular Structure Based on Hyaluronic Acid Derivatives for the Preparation of Vascular and Urethral Graft

(76) Inventors: Giovanni Abatangelo, Saccolongo (IT); Lanfranco Callegaro, Thiene (IT)

Correspondence Address: ABELMAN, FRAYNE & SCHWAB, 666 THIRD AVENUE, 10TH FLOOR, NEW YORK, NY, 10017, US

(21) Appl. No.: 11/666,200

(22) PCT Filed: Oct. 27, 2005
(86) PCT No.: PCT/EP05/55610

§ 371 Date: Apr. 23, 2007

(30) Foreign Application Priority Data
Oct. 27, 2004 (IT) PD2004A000265

(51) Int. Cl. A61F 2/02

(52) U.S. Cl. 424/423; 523/113

(57) ABSTRACT

Tubular structure, whose wall has an unbroken surface consisting essentially of at least one HA derivative and optionally a further polymer of natural, synthetic or semisynthetic origin. Said tubular structure, which is prepared with a very simple process, is used for the preparation of vascular and urethral grafts.

### FIELD OF THE INVENTION

The present invention is a tubular-shaped biomaterial, comprising a hyaluronic acid derivative, that is able, when used as a vascular graft, to induce guided vascular regeneration after being implanted in vivo, that leads to the de novo reconstitution of the vascular wall of small and medium-sized arteries.

### BACKGROUND OF THE INVENTION

Cardiovascular disorders with atherosclerotic complications (ASCVD, AtheroSclerotic CardioVascular Diseases) constitute the most common class of pathology worldwide. The most frequent are coronary disorders, infarct, ictus and arterial hypertension. Their incidence and prevalence in the population are constantly on the increase, as a result of both unhealthy lifestyle and the lengthening of the average lifespan. The prevention, cure and management of these pathologies is extremely costly; the calculated direct and in direct costs for 2004 in the United States amount to 370 million dollars (Heart Disease and Stroke Statistics 2004 Update, American Heart Association, Dallas, Tex.). Treatment of these diseases is, however, a priority of public health spending. Despite the constant progress being made in endovascular surgery, the favoured approach to therapy for coronary and/or peripheral occlusions remains the surgical implantation of prostheses to create a circulatory by-pass. This procedure can be applied to lesions in a wide variety of anatomical sites and ensures a good degree of patency to the vessel in the long term. In the case of larger vessels (diameter>6 mm) synthetic prostheses made of materials such as Dacron®, Polytetrafluoroethylene or Polyurethane are used successfully. Although they have brought about a reduction in the need for repeat surgery, these materials do tend to cause infection around the suture, to give rise to occlusions and to become dilated (Conte, Faseb J; 1998; 12:43-45), so there is a considerable risk of side effects when they are used.

The abovesaid materials are not indicated for use in small vessels (diameter 3-5 mm) such as the coronary and carotid arteries, because they are not sufficiently elastic to withstand the low blood flow rate. In this type of surgery, therefore, grafts of autologous vessels are usually used (such as a saphenous vein, internal mammary artery or radial artery).

The results obtained are good both in terms of patency of the vessel and elasticity of the graft, but they tend to be short-lived, probably because as the grafted vessel adapts to the new blood flow, it undergoes hyperplasia of the intima, not only at the point where it is stitched but also along its length. This determines stenosis that drastically reduces the blood flow, leading to failure of the graft itself (ibidem). Moreover, the availability of these materials may prove insufficient in patients requiring multiple grafts because of diffuse vascular disorders.

These factors together have led researchers to find investigate tissue engineering techniques by which it is possible, using a multidisciplinary approach, to create graft structures (such as cardiac valves and blood vessels) that are viable and completely autologous. Since they are viable, the bioengineered blood vessels are sensitive to stimuli and are self-renewable, with an intrinsic capacity for healing and remodelling according to the requirements of the specific environment in which they are implanted. Generally speaking, tissue engineering of the blood vessels starts with a supporting structure or scaffold constituted by a natural or synthetic bioresorbable material. The scaffolds provide a temporary biomechanical support until the endothelial cells of the original vessel have themselves produced extracellular matrix. Various kinds of scaffold have been used to date, such as:
biological scaffolds for example decellularised matrices, the use of which is however limited by the risk of viral infections;
non-biodegradable polymers (Dacron®, Polytetrafluoroethylene) which perform well in vivo, but are not very suitable for small vessels for the reasons set forth above;
polyurethane, which initially presents good biocompatibility but then undergoes chemical modification on its surface and consequent rapid degradation;
polyglycolic and polylactic acids, which have, however, only been used in experimental trials.

Each of these materials has a different performance profile according to its individual characteristics, but, to date, the most serious limitations to their use in vivo concern:
relatively high thrombogenicity, due to the inability of the material to mimic the mechanical properties of the native artery;
inability to enable a rapid and complete regeneration of the endothelial layer (Mitchell et al., Cardiovasc Pathol, 2003; 12:56-64; Moldovan et al., Arch Pathol Lab Med, 2002; 126:320-324;)
high degree of degradation in vivo and subsequent triggering of an acute inflammatory action (Greisler et el., Arch Surg, 1982; 117:1425-1431; Santavirta et al., J Bone Jt Surg Br, 1990; 72:597-600).

One natural polymer presents a particularly interesting profile, however: hyaluronic acid (HA), chemically modified so as to obtain three-dimensional matrices to be used as biomaterials for the preparation of new engineered tissues. HA is a hetero-polysaccharide composed of alternating residues of D-glucuronic acid and N-acetyl-D-glucosamine; it is a straight-chain polymer with a molecular weight varying between 50,000 and 13×10⁶ Da, according to the source it was obtained from and the methods used to prepare it. It is present in nature in the pericellular gels, in the fundamental substance of the connective tissue of vertebrates, in the synovial fluid of joints, in the vitreous humor and in the umbilical cord. Since it is practically ubiquitous, HA plays an important biological role in the organism, especially as a mechanical support for the cells of many different tissues (skin, tendons, cartilage, muscles); it is also well known that, through its CD44 membrane receptor, HA modulates numerous different processes relating to cell physiology and biology, such as cell migration and differentiation and angiogenesis, and is responsible for tissue hydration and joint lubrication.

The chemical modifications performed on the HA molecule, known to the state of the art to be the most interesting for the obtainment of biomaterials are:
salification of HA with organic and/or inorganic bases (EP 138572 B1);
esterification of HA (HYAFF®) with alcohols of the aliphatic, araliphatic, cycloaliphatic, aromatic, cyclic and heterocyclic series (EP 216453 B1);
inner esterification of HA (ACP®): inner esters of HA with esterification not exceeding 20% (EP 341745 B1);
amidation of HA (HYADD™) with amines of the aliphatic, araliphatic, cycloaliphatic, aromatic, cyclic and heterocyclic series (patent application No. EP 1095064);
deacetylation of HA on the N-acetyl-glucosamine fraction (patent application EP 1313772);
O-sulphatation of HA (EP 702699 B1);
percarboxylation of HA (HYOXX™) by oxidation of the primary hydroxyl of the fraction of N-acetyl-D-glucosamine (patent application EP 1339753).

Of the numerous derivatives listed above, those particularly suitable for the formation of new engineered tissues have proved to be the HA esters, and especially so the benzyl ester (HYAFF®11), as demonstrated for example by Campoccia et al. (Biomaterials, 1998,19:2101-2127).

Moreover, experiments known to the state of the art (Turner N J, et al., Biomaterials, 2004, 25: 5955-5964) reveal that endothelial cells taken from human saphenous veins are able to proliferate perfectly on scaffolds constituted by HYAFF®11 fibres worked into a non-woven mesh (EP 618817 B1), once seeded thereon in suitable conditions which an expert in the field would be well acquainted with. The cells first adhere to the fibres of HYAFF®11 and then proliferate inside the fibrous mesh, spreading through the interstices until, within an interval of about 20 days, they form a compact monolayer over the surface of the scaffold that is characterised by a well-organised subendothelial matrix. A first attempt to create small blood vessels was made by Remuzzi A. et al. (Tissue Eng, 2004, 10: 699-710) who first grew smooth-muscle, vascular cells obtained from porcine thoracic aorta on a non-woven, totally esterified HYAFF®11 mesh (HYAFF®-11 p100) and then wrapped the mesh round cylindrical silicone supports, stitched it in place and left the cells to grow for another 14 days. At the end of this process, the silicone support is pulled out, leaving cylinders with an outer diameter of about 6 mm and an inner diameter of about 4 mm, the outer surface of which presents a fair extracellular matrix, while the muscle cells are to be found inside the HYAFF®11 scaffold. Although its consistency is right and it is completely biocompatible, this type of structure is not suitable for the purposes of the present invention because it is not able to bear the pressure of the blood flow. Indeed, mechanical resistance tests have shown that they are decidedly less resistant than the starting porcine coronary vessels, probably because the various layers of rolled HYAFF®11 do not completely adhere to each other, so that the tube is of uneven thickness; moreover, inside the cylinder the layer of endothelial cells is insufficient and the layer of smooth muscle cells is not continuous. A combination of these elements is vital to the mechanical stability and functional efficiency of a vascular graft. Inventions that are already known to the state of the art describe tubular structures of HYAFF®11, in which the HYAFF®11 cylinder is enriched with a single thread wound round it in the form of a helix (EP 571415 B1), or with several threads made of the same material knitted together (EP 652778 B1) and inserted inside the tube to add to its compactness. These scaffolds have been used successfully in the regeneration of nerve fibres. Other tubular HYAFF®11 structures have been used for the regeneration of the urethra (Italiano G. et al., Urol Res, 1997, 26:281-284): in this case the tubes were formed by a mesh of HYAFF®11 fibres.

### SUMMARY OF THE INVENTION

The present invention goes way beyond the limits of the current know-how of an expert in the field. It relates to a new tubular structure, whose wall has an unbroken surface, consisting essentially of at least one HA derivative and optionally a further polymer of natural, synthetic or semisynthetic origin.

These tubular structures enable the complete reconstruction of the vessel wall when grafted directly in vivo. Moreover, they are biocompatible, biodegradable and adapt perfectly to the physiology and blood dynamics of the district wherein they are implanted, constituting an excellent tubular join. Their characteristics enable them to enhance the regeneration of the walls that constitute the urethra and their use is therefore justified in uro-genital surgery.

Therefore the present invention further relates to:
a vascular graft comprising the tubular structure according to the present invention;
urethral graft comprising the tubular structure according to the present invention.

Finally the present invention further relates to a process for preparing said tubular structure comprising the following steps:
(I) dissolving the HA derivative in DMSO and optionally the second further polymer;
(II) coating with the solution thus obtained rotating steel cylinders of varying diameters;
(III) coagulating the solution adhered to the cylinder in an ethanol bath;
(IV) removing from the cylindrical support, washing with ethanol and air blow drying the tubular structure,
(V) cutting, packaging and sterilising by ? ray the tubular structures thus obtained.

### DETAILED DESCRIPTION OF THE FIGURES

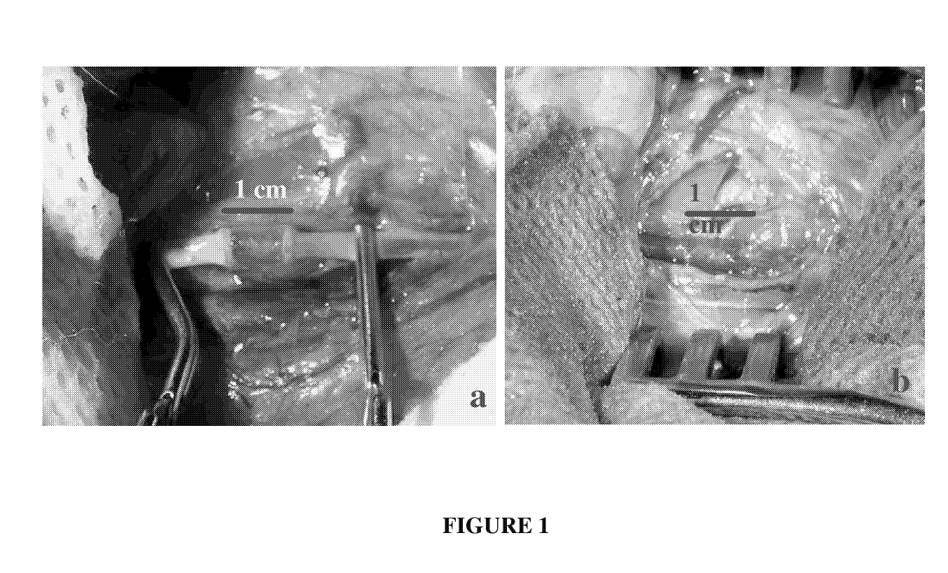

FIG. 1 reports two photos of the tubular structure (guide channel) according to the present invention of HYAFF®11p100 (diameter 2 mm, length 1 cm) anastomosed in the abdominal aorta, (a) before and (b) after release of the vascular clamp.

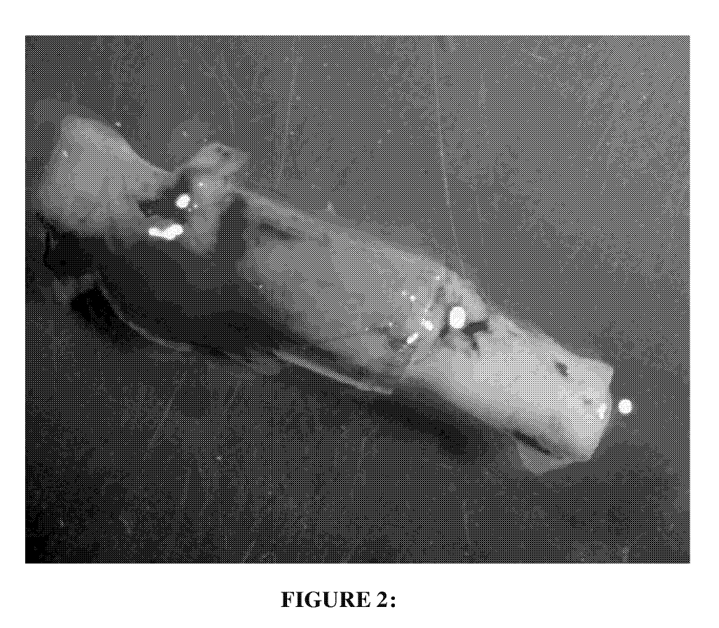

FIG. 2 reports the photo of a specimen of HYAFF®11p100 (diameter 2 mm, length 1 cm) anastomosed in the abdominal aorta, recovered 15 days after implantation. The tubular structure has maintained its mechanical properties and shows no signs of dilatation. The regenerated artery is already clearly visible inside the guide channel.

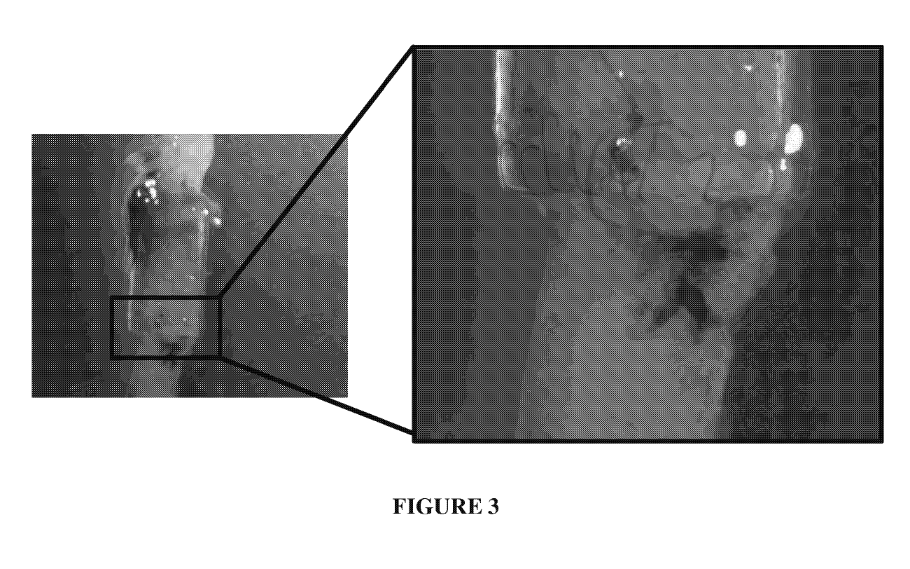

FIG. 3 reports magnified photos of the appearance of the HYAFF®11p100 guide channel already represented in the previous photo recovered 15 days after implantation: the stitches bear the longitudinal tension and maintain axial and radial flexibility and pulsation until the vessel has completely regenerated. The specimen is free moving and there are no fibrous adhesions to the surrounding tissues.

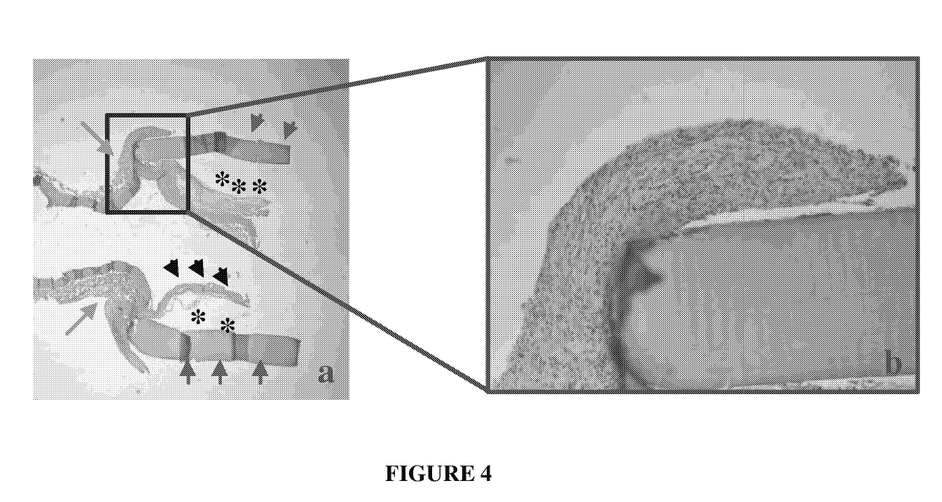

FIG. 4: (a) longitudinal section of the specimen (haematoxylin-eosin, magnified 5×) on the 5^{th} day: the blue arrows point to the endothelial layer that is being formed; the green arrow point to the anastomosis site, where the aorta comes into contact with the implanted guide channel; the red arrows point to the HYAFF®11 p100 guide channel; the asterisks indicate an absence of any infiltration of the vascular tissue into the biomaterial. The regenerative process is ongoing inside the guide channel.

(b): the same specimen magnified 20×: the aorta comes into contact with the guide channel at the point of anastomosis.

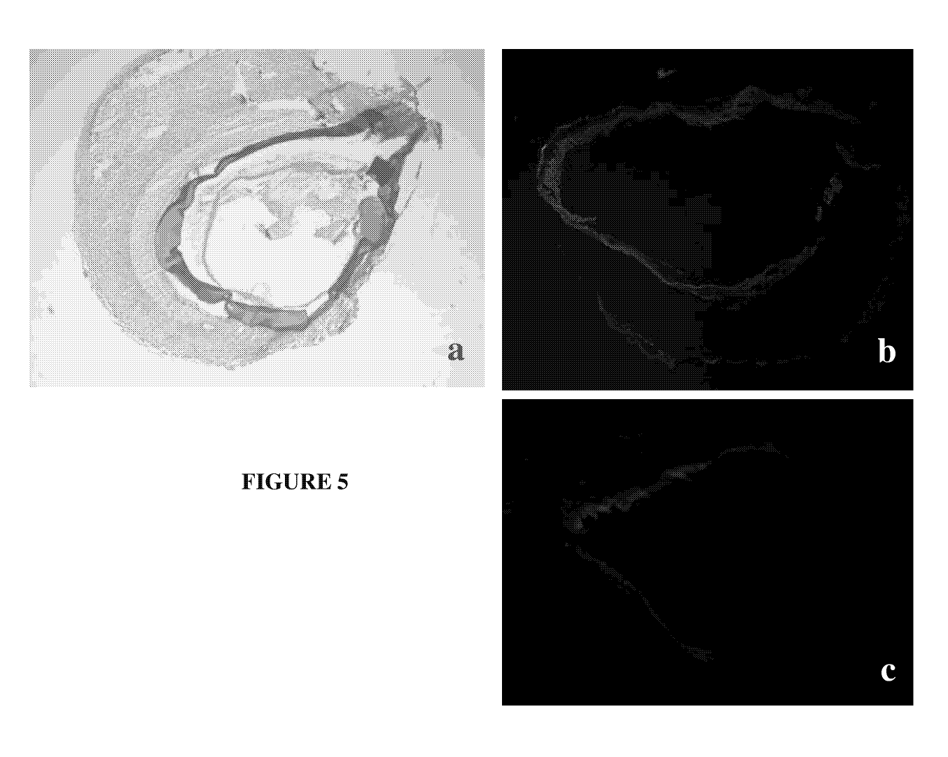

FIG. 5: (a) reports a photo of the cross section of a specimen (haematoxylin-eosin, magnified 2.5×) on the 5^{th} day. (b): (antihuman von Willebrand factor antibodies magnified 5×) confirms the presence of a well represented endothelial layer. (c) Immunofluorescence analysis (Antimyosin Light Chain Kinase antibodies, magnified 5×) shows the beginning of an as yet indistinct smooth muscle component.

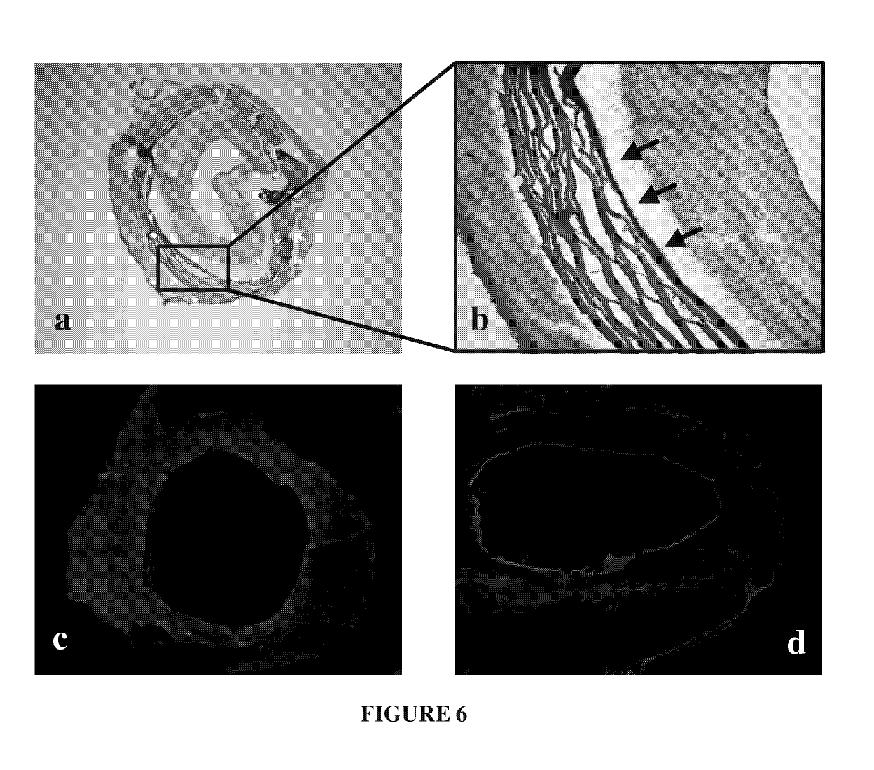

FIG. 6: (a) reports a cross-section of a specimen (Weighert, magnified 2.5×) on the 15^{th} day: all the vessel walls are well represented. (b) The tubular structure is still present and has maintained its mechanical properties (Weighert magnified 10×); (c) and (d) respectively show the layer of smooth muscle cells (anti-Myosin Light Chain Kinase antibodies 5×) and the endothelial layer (antihuman von Willebrand factor antibodies, 5×)

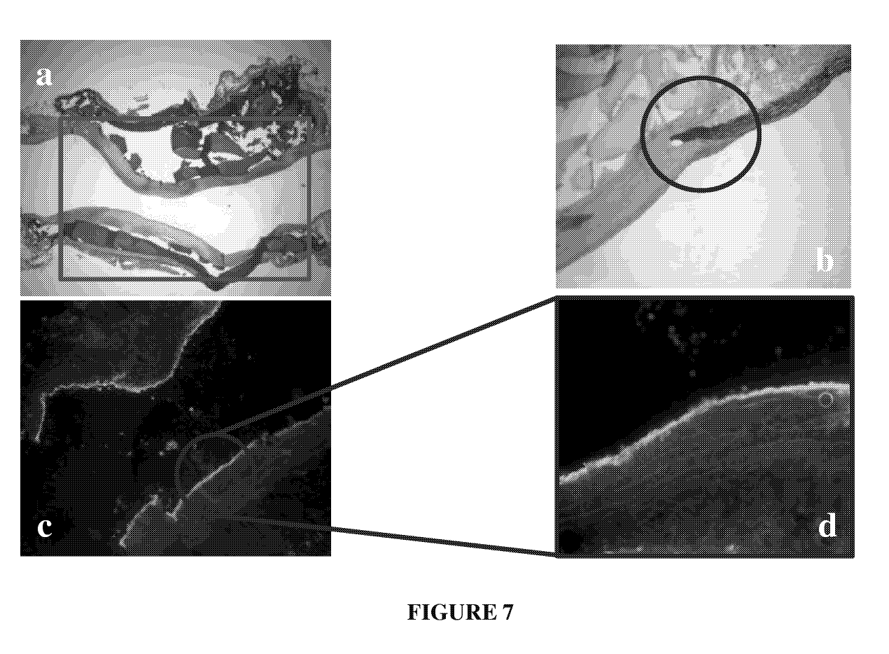

FIG. 7: (a) longitudinal section of specimen (Weighert magnified 2.5×) on the 30^{th} Day: the blue rectangle indicates the stretch of new artery; there are no signs of occlusion, dilatation or collapse of the vessel walls. The biomaterials appears to have crumbled into fragments, as a results of difficulty when cutting it. (b) Site of anastomosis, magnified 10×: the original artery walls connect with the newly formed section. (c) and (d) respectively show 5× and 10× magnifications of the endothelial layer (anti-human von Willebrand factor antibodies).

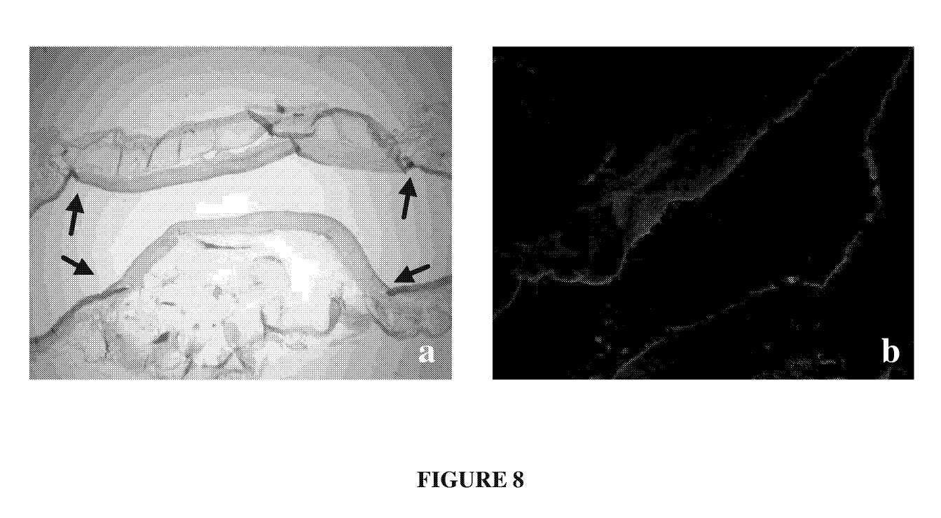

FIG. 8: (a) longitudinal section of specimen (Weighert, magnified 2.5×) on the 60^{th} day: the blue arrows point to the area of transition between the original artery and the newly formed section.(b) The endothelial layer coats the entire surface of the lumen of the newly formed artery (immunofluorescence with anti-human von Willebrand factor antibodies), magnified 5×.

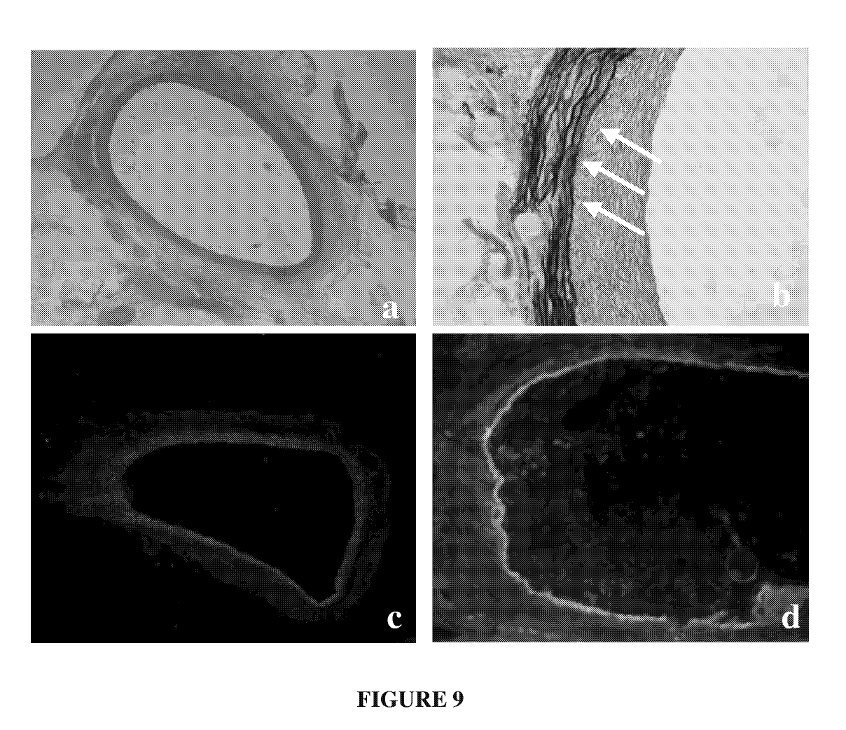

FIG. 9 (a) cross section (haematoxylin-eosin, magnified 5×) on the 60^{th} day: all the components of the vessel are well represented. (b) Cross section (Weighert, magnified 20×): the elastic element is clearly visible. (c) Immunofluorescence (anti-Myosin Light Chain Kinase antibodies, magnified 5×) confirms the presence of smooth muscle cells. (d) Immunofluorescence with anti-human von Willebrand factor antibodies (magnified 10×) shows a coating of endothelial cells.

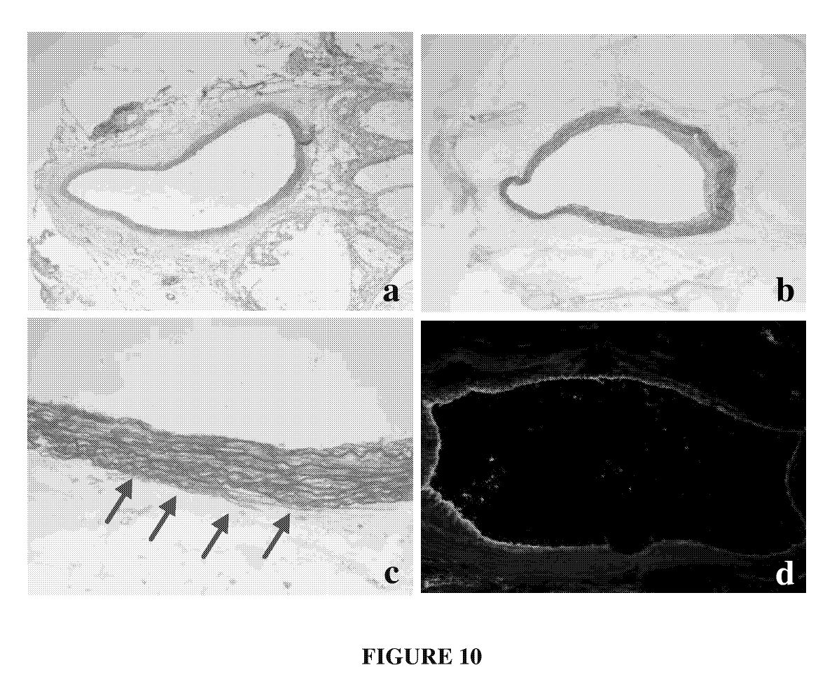

FIG. 10: (a) cross section (haemotylin-eosin, magnified 5×) on the 60^{th} day: the biomaterial has disappeared. (b) cross-section (Weighert, magnified 5×), the elastic component is well represented. (c) Cross section (Weighert, magnified 40×): details the elastic component (blue arrows). (d) Immunofluorescence with antihuman von Willebrand factor antibodies (magnified 10×): the layer of endothelial cells is clearly visible.

### DETAILED DESCRIPTION OF THE INVENTION

For the purpose of the present invention the definition that the tubular structure “consists essentially of at least one HA derivative and optionally a further polymer” means that the at least one HA derivative optionally associated to a second polymer is present in said tubular structure in total amounts=95%, by weight based on the total weight of the tubular structure.

In the present invention the HA derivatives preferably used for preparing the tubular structure according to the present invention are selected from HA esters with alcohols of the aliphatic, araliphatic, cycloaliphatic, aromatic, cyclic and heterocyclic series (HYAFF®), amides of HA with amine of the aliphatic, araliphatic, cycloaliphatic, aromatic, cyclic and heterocyclic series (HYADD™), deacetylated, O-sulphatated and percarboxylated HA derivatives, and mixtures thereof.

More preferably the hyaluronic acid derivatives are hyaluronic acid esters. Even more preferably the hyaluronic acid ester are selected from those whose carboxy functions have been esterified with benzyl alcohol (HYAFF®11) with between 50 and 100% esterification degree.

According to particularly preferred embodiments of the present invention the hyaluronic acid benzyl esters used for the purpose of the present invention have an esterification degree of from 75 to 100%.

The tubular structures according to the present invention can be used above all as temporary ducts in vascular surgery to the small and medium sized arteries. By the examples and experiments described in detail hereafter the Applicant has demonstrated that the structures described herein have all the mechanical and functional characteristics necessary for the set purpose, because they:
are biocompatible with the biological fluids; while they remain in situ there is no evidence of infiltration by monocytes or neutrophils, cells that are typically present in the phase of inflammatory response to the presence of a foreign body;
are biodegradable, because they are degraded by the macrophages within 4 months of implantation. Observation after 120 days reveals a biomaterial that looks like a gel to the naked eye, indicating that the degradation process is complete, and that the degradation products do not trigger any kind of inflammatory response;
have sufficient mechanical resistance to sustain the flow of blood, simulating the physiological behaviour of the natural arteries, thus preventing the formation of abnormal dilations, typical of implants constituted by other materials;
they are not thrombogenic; it is known that acute thrombosis can be caused by the blood coming into contact with structures other than the endothelium. In the experiments described hereafter there was no evidence of the formation of thrombi;
they ensure uniform regeneration of both the vascular and urethral walls, enabling total recovery of the function of the duct wherein they have been implanted; indeed, the grafts described herein induce a sequential growth of the vascular wall, mimicking the physiological arteriogenic process;
they isolate the regenerative process from the surrounding environment, thus avoiding any possible negative interference, and the surrounding tissues do not adhere to them;
they are compact and consistent enough to be easily handled and stored, and require only straightforward suture during surgery;
they can be made in various different lengths and diameters, as their production process is very simple.

The prosthesis obtained with the tubular structure according to the present invention, thanks to the intrinsic properties of the material used (preferably HYAFF®11), provides a solution to all the limitations encountered to date, and represents a breakthrough in the field of uro-genital and vascular surgery, especially for vessels measuring between about 2 and 5 mm (coronary, internal carotid, brachial, posterior tibial arteries), between 7 and 10 mm circa (common carotid artery, popletial artery, common iliac and common femoral arteries). It can also be applied to larger vessels, (such as the abdominal and thoracic aortas). Materials suitable for the purposes of the present invention can also be obtained from an HA derivative associated with an other type of HA derivative and/or other natural, semisynthetic or synthetic polymers. Preferably natural polymers include: collagen, elastin, coprecipitates of collagen and glycosaminoglycans, cellulose, polysaccharides in the form of a gel, such as chitin, chitosan, pectin or pectic acid, agar, agarose, xanthane gum, gellan, alginic acid or alginates, polymannan or polyglycans, polyamides, natural gums.

Preferably semisynthetic polymers include:
collagen cross-linked with agents selected from aldehydes or the precursors thereof, dicarboxylic acid or the halides thereof and diamines,
derivatives of: cellulose, alginic acid, starch, chitin, chitosan, gellan, xanthane, pectin or pectic acid, polyglicans, polimannan, agar, agarose, natural gums, glycosaminoglycans.

Lastly, preferred synthetic polymers include polylactic and polyglycolic acids, or copolymers or derivatives or derivatives thereof, polydioxane, polyphosphazene, resins.

More preferably the tubular structure according to the present invention, in case they contain a second polymer of semisynthetic origin, this is selected from an ester of carboxy-methylcellulose more preferably the benzyl ester, or an ester of alginic acid, more preferably the benzylester.

The weight ratio of hyaluronic acid derivative/other polymer, in case the latter present, is preferably comprised between: 95:10 and 60:40.

More preferably the weight ratio hyaluronic acid derivative/other polymer is comprised between 80/20 and 70/30

According to a particularly preferred embodiment the tubular structure in case it contains an other polymer is formed by hyaluronic acid benzyl ester 100% (HYAFF®11p100) and benzyl ester of carboxymethylcellulose in weight ratio 80/20.

According to an other particularly preferred embodiment the tubular structure according to the present invention, in case it contains an other polymer, it consists of (HYAFF®11p100) and benzyl ester of alginic acid in weigh ratio of 70/30.

It is also possible to prepare said tubular structures associating the HA derivatives with one or more pharmacologically and/or biologically active substances. In the process according to the present invention in step (I) the concentration in DMSO of hyaluronic acid and the optional second polymer is preferably comprised between 70 and 160 mg/ml, more preferably between 80 and 150 mg/ml.

Preclinical Research

For purely descriptive purposes and without being limited to the same, we report hereafter some examples of the preparation of grafts that are the subject of the present invention, and the results obtained from in vivo experiments which demonstrated the absolute efficacy and safety of the materials claimed in the present invention.

Preparation of Tubular Prostheses Made with the Total Benzyl Ester of HA

The total benzyl ester of HA (HYAFF®-11 p100) was dissolved in dimethylsulphoxide (DMSO, 80-150 mg/ml) and the solution of HYAFF/DMSO was used to coat a rotating cylindrical steel bar with a diameter varying between 1 and 10 or more mm, according to the type of duct to be regenerated. The solution of HYAFF/DMSO coated on the cylinder was then coagulated in an ethanol bath. The tube thus formed was gently removed from around the cylinder, cut into suitable portions, washed in ethanol and air-blown dry. The prostheses obtained by this procedure were packed in double packs and sterilised with ? rays.

Preparation of Tubular Prostheses with the Benzyl Ester of HA and the Benzyl Ester of Carboxymethylcellulose

A mixture of powders composed of the total benzyl ester of HA (HYAFF®-11 p100) and a benzyl ester of carboxymethylcellulose in a ratio of 80/20 is dissolved in DMSO at a concentration of 100 mg/ml. Once solubilisation is complete, the mixture is treated as described in Example 1.1.

Preparation of Tubular Prostheses with the Benzyl Ester of HA and the Benzyl Ester of Alginic Acid

A mixture of powders composed of the total benzyl ester of HA (HYAFF®-11 p100) and a benzyl ester of alginic acid in a ratio of 70/30 is dissolved in DMSO at a concentration of 120 mg/ml. Once solubilisation is complete, the mixture is treated as described in Example 1.1.

1.4 Implantation of the Prostheses

For the experiments described hereafter, 30 male, Wistar rats weighing 250-350 g were anaesthetised by the intraperitoneal route with a cocktail of ketamine hydrochloride 40 μg e xylazine 20 μg/100 mg in weight. The abdominal area was shaved and rendered aseptic with Betadine and 70% alcohol. The abdominal muscles were exposed through an incision of about 3 cm; the part of the aorta between the renal arteries and the aortic trifurcation was then exposed. Once the vessel had been clamped, a segment of aorta of 1 cm was incised and a tube of HYAFF®-11 p100 (diameter 2 mm, length 1 cm, prepared as per Example 1.1) was inserted by anastomosis, first proximally and then distally, and then stitched with continuous suture using nylon 10.0 thread (FIG. 1). No anticoagulants were used either before or after surgery. All surgical procedures were performed in the same way and by the same person.

1.5 Collection of the Prostheses

At each time point (5, 15, 30, 60 and 120 days) 5 animals were sacrificed. The graft area was carefully exposed through the earlier access incisions. After clamping, the aorta was incised transversally starting from the distal end, to 3 mm from the site of anastomosis, and the segments thus obtained were thoroughly rinsed in heparin saline solution (NaCl 0.9%) (FIG. 2). The resulting segments were then cut transversally in half and fixed separately in formaldehyde and a specific medium for frozen tissue samples (O.C.T. Tissue-Tek®), for histological and immunohistochemical analysis respectively. Some whole samples were fixed in formalin, embedded in paraffin and then cut into lengthwise sections for staining.

1.6 Histological Analysis

The samples fixed in formaldehyde were gradually dehydrated in ethyl alcohol, embedded in paraffin and then cut along the longitudinal axis of the sample into sections 7 μm thick, which were then stained with haemotoxylin eosin (HE) and Azan-Mallory stain for histological tests, while Weighert's stain revealed the presence of elastin fibres.

1.7 Immunofluorescence Analysis

The endothelial cells were characterised by assessing the intracellular expression of the von Willebrand factor (Factor VIII): the samples previously placed in OCT were frozen in liquid nitrogen and then cut with a cryostat into 5 μm-thick sections. The immunofluorescence studies were conducted using polyclonal antibodies (produced in rabbit) human von Willebrand anti-factor, diluted 1:300 (DAKO); after 1 hour of incubation, the samples were rinsed with saline and treated with anti-polyclonal secondary antibody bound to a fluorescent pigment (TRICT).

The smooth muscle cells were identified and characterised, measuring the expression of Myosin Light Chain Kinase (MLCK), according to the method described by Vescovo et al. (BAM; 1996; 6:183-187).

2. Preclinical Data

2.1 Macroscopic Observations on Implantation and Collection

From a surgical point of view, the tubes of HYAFF®-11 p100 appeared soft and elastic, easy to cut and stitch and with ideal characteristics for suture of the anastomosis with 10.0 nylon thread. It took about 50 minutes to complete the two anastomoses, as reported in the literature (Zhang et al., Biomaterials; 2004; 25: 177-187). Once the blood flow had been restored through the prosthesis, dilation of the pulse was visible to the naked eye and the slight bleeding from the tube was easily stemmed with a gauze pad (FIG. 1b). When the prostheses were recuperated, no signs of thrombosis and/or infection were visible around the graft (FIGS. 2 and 3); also absent were any signs of aneurismatic dilation or collapse of the vessel walls. The mechanical properties of the vascular duct remained intact until complete regeneration of the arterial segment (FIG. 3). The survival rate of the animals was 100%, and there was no manifestation of vascular failure in the peripheral districts.

2.2 Group 1 (5 Days)

The data relative to the observations made on the 5^{th} day are shown in FIGS. 4 and 5; the anastomoses are solid and well integrated with the original artery (FIG. 4a) and the tubes of HYAFF®-11 p100 maintain their original chemical and mechanical characteristics (FIG. 5a). The endothelial coating begins to regenerate both proximally and distally with regard to the anastomosis, it runs inside the prosthesis without any sign of infiltration and tends to converge at the middle. At the same time, a temporary tissue develops from the aorta and wraps around the outside of the vessel duct at the suture sites (FIG. 4b). Immunofluorescence analyses confirm the presence of endothelial cells (FIG. 5b) and reveal the early stages of the formation of a thin layer of smooth muscle cells inside the duct (FIG. 5c).

2.3 Group 2 (15 Days)

On the 15^{th} day, the arterial tract is completely regenerated and all the vascular structures are well represented and organised, as shown in FIG. 6. The tube is still present (blue arrows in FIG. 6b) and, as demonstrated by immunofluorescence of a transversal section (FIG. 6d), the endothelial layer entirely coats the lumen of the graft. The presence of smooth muscle cells is also clearly evident (FIG. 6c), as well as extracellular matrix components (collagen and elastin), normally produced by smooth muscle cells in the median area of the arterial wall. Collagen and elastin give the newly-formed vessel sufficient mechanical resistance for it to withstand suture and avoid breakage.

2.4 Group 3 (30 Days)

On the 30^{th} day, the HYAFF®-11 p100 tube is still present and the newly-formed artery runs inside it. Histological analysis of the samples (FIGS. 7a and 7b, staining with Haematoxylin-Eosin) clearly reveals that the new vessel walls are well integrated with the original artery at the site of anastomosis. Immunofluorescence confirms the presence of the endothelial coating (FIGS. 7c and 7d).

2.5 Group 4 (60 Days)

On the 60^{th} day the prosthesis is still present and the regenerative process proceeds normally. Endothelial and smooth muscle cells are clearly visible (FIGS. 8b, 9c, 9d). The walls of the new artery are stratified like those of a normal vessel (FIGS. 8a and 9a) and the elastic component is very evident (FIG. 9b). All the vascular structures are therefore organised and on histological analysis appear identical to those of any ordinary arterial tract.

2.6 Group 6 (120 Days)

The most important finding at this point of the study is the absence of the biomaterial revealed by histological tests (FIGS. 10a and 10b). The new artery maintains its original mechanical and structural characteristics. The lumen is patent and shows no signs of dilation or collapse. Weighert's stain confirms the presence of a mesh of elastic fibres (FIG. 10c), while the endothelial layer is again detected by immunofluorescence (FIG. 10d).

From the above account, it can therefore be deduced that the new tubular structures that are the subject of the present invention, constituted preferably by hyaluronic acid esterified with benzyl alcohol (HYAFF®11) with 100% esterification, have all the fundamental requisites to be considered, to all effects, systems for assisted vascular and/or urethral regeneration, to be used directly in vivo. Indeed, because of the peculiar character of the biomaterial used, the tubes claimed herein are biocompatible, biodegradable and therefore temporary, capable of allowing the fast and normal growth of vascular and/or urethral tissues and of becoming perfectly integrated with the environment wherein they are implanted, both from a functional and mechanical point of view, until the damaged structure has been completely regenerated. The tool claimed herein is therefore new, safe, easy to make and handle, able to solve any problem linked with the implantation of vascular and/or urethral replacements used to date in clinical practice. The invention therefore constitutes an enormous step forward in the surgical treatment of cardiovascular diseases with atherosclerotic complications. The invention being thus described, it is clear that the examples for the preparation of the biomaterial in question can be modified in various ways. Such modifications are not to be considered as divergences from the spirit and purpose of the invention, and any modification that would appear evident to an expert in the field comes within the scope of the following claims.

1. A tubular structure suitable for use as vascular or urethral grafts, whose wall has an unbroken surface, consisting essentially of at least one hyaluronic acid derivative, optionally in association with at least one other polymer of natural synthetic or semisynthetic origin.

2. The tubular structure according to claim 1, consisting essentially of at least one hyaluronic acid derivative.

3. Tubular structure according to claim 1 wherein the hyaluronic acid derivative is chosen from a group consisting of: hyaluronic acid esters with alcohols of the aliphatic, araliphatic, cycloaliphatic, aromatic, cyclic and heterocyclic series; amides of hyaluronic acid with amines of the aliphatic, araliphatic, cycloaliphatic, aromatic, cyclic and heterocyclic series; deacetylated hyaluronic acid; O-sulphated hyaluronic acid; percarboxylated hyaluronic acid, and mixtures thereof.

4. The tubular structure according to claim 3 wherein the hyaluronic acid derivative is an ester.

5. The tubular structure according to claim 4 wherein the hyaluronic acid ester is preferably the benzyl ester with a degree of esterification of between 75 and 100%.

6. The tubular structure according to claim 5, wherein the hyaluronic benzyl ester has an esterification degree of 100%.

7. The tubular structure according to claim 1, wherein said further optional polymer is of natural origin and it is selected from the group consisting of: collagen, elastin, coprecipitates of collagen and glycosaminoglycans, cellulose, polysaccharides in the form of a gel, selected from chitin, chitosan, pectin or pectic acid, agar, agarose, xanthane gum, gellan, alginic acid or alginates, polymannan or polyglycans, polyamides, natural gums.

8. The tubular structure according to claim 1, wherein said further optional polymer is of semisynthetic origin and it is selected from the group consisting of:
collagen cross-linked with agents selected from aldehydes or the precursors thereof, dicarboxylic acid or the halides thereof and diamines,
derivatives of: cellulose, alginic acid, starch, chitin, chitosan, gellan, xanthane, pectin or pectic acid, polyglicans, polymannan, agar, agarose, natural gums, glycosaminoglycans.

9. The tubular structure according to claim 1, wherein said further polymer is of synthetic origin and it is selected from polylactic and polyglycolic acids, copolymers and derivatives thereof, polydioxane and polyphosphazene resins.

10. The tubular structure according to claim 1, having a weight ratio of hyaluronic acid derivative/further optional polymer, in case the latter present, comprised between: 95:5 and 60:40.

11. The tubular structure according to claim 1, further containing at least one pharmacologically and/or biologically active substance.

12. A vascular graft comprising the tubular structure according to claim 1.

13. An urethral graft comprising the tubular structure according to claim 1.

14. A process for preparing the tubular structure according to claim 1 comprising the following steps:
(I) dissolving the HA derivative in DMSO and optionally the second further polymer;
(II) coating with the solution thus obtained rotating steel cylinders of varying diameters;
(III) coagulating the solution adhered to the cylinder in an ethanol bath;
(IV) removing from the cylindrical support, washing with ethanol and air blow drying the tubular structure,
